(19) United States
(12) Patent Application Publication
(10) Pub. No.: US 2017/0156297 A1
(43) Pub. Date: Jun. 8, 2017

(54) ANIMAL MODEL FOR STUDYING NEUROBLASTOMAS

(71) Applicants: UNIVERSITE CLAUDE BERNARD LYON 1, Villeurbanne Cedex (FR); CENTRE NATIONAL DE LA RECHERCHE SCIENTIFIQUE, Paris Cedex 16 (FR)

(72) Inventors: Valérie CASTELLANI, Villeurbanne (FR); Céline DELLOYE-BOURGEOIS, Lyon (FR)

(21) Appl. No.: 15/323,986

(22) PCT Filed: Jul. 7, 2015
(86) PCT No.: PCT/EP2015/065509

§ 371 Date: Jan. 5, 2017

(30) Foreign Application Priority Data
Jul. 7, 2014 (FR) 1456531

(51) Int. Cl. A01K 67/027; C12N 5/26; G01N 33/574; A61K 49/00

(52) U.S. Cl. CPC A01K 67/0271; A61K 49/0008; C12N 5/166; G01N 33/57407; A01K 2227/30; A01K 2207/12; A01K 2267/0331

(57) ABSTRACT

The present invention relates to a gallinaceous bird embryo into which human neuroblastoma cells have been grafted at the level of the neural crests. The invention also relates to a process for preparing such a gallinaceous bird embryo, and to a process for screening for therapeutic molecules intended for the treatment of a neuroblastoma tumor based on the use of this gallinaceous bird embryo.

### FIELD OF THE INVENTION

The present invention relates to an animal study model for neuroblastoma, which is a pediatric solid tumor representing 10% of the cancers in children.

### PRIOR ART

Neuroblastoma (NB) is a pediatric tumor disease. The average age of discovery in children is between one and two years old. Its instance is approximately one case per 100 000 children, and very rare cases have been identified in adults. The progression of the disease is very variable, the forms with isolated tumor having a much better prognosis than the forms with metastases. The cause of its occurrence is unknown, the vast majority of cases being sporadic and non-familial.

This tumor derives from embryonic neural crest neuroblasts (NCCs) and develops mainly in the sympathetic ganglia and the adrenal gland medullae. The neural crest denotes a population of multipotent cells generated from the most dorsal region in the neural tube. These cells migrate in the whole of the embryo during development and give rise to a large variety of cell types in the adult. Among these, the neuroblasts develop into neurons.

The embryonic microenvironment within which neuroblastomas emerge is specific and cannot be studied on the majority of animal tumorigenesis models, in particular mice and rats, which limits the possibilities of preclinical tests of candidate therapeutic molecules.

Genetically modified animal models, which make it possible to study the role of the amplification of the MYCN or ALK oncogenes, commonly observed in human neuroblastomas, have been described: they are mice that have been genetically modified to express MYCN (Weiss et al., 1997) or zebrafish overexpressing MYCN and/or ALK (Zhu et al., 2012).

However, the MYCN and/or ALK oncogenes are not overexpressed in all types of neuroblastomas observed in patients.

At the current time, there is no animal model for all the forms of neuroblastoma, in which tumors would develop in an animal body according to the same configuration as in the human body, namely in the sympathetic ganglia and the adrenal gland medullae. Such a model is, however, necessary in order to gain a more detailed knowledge of the disease, and also to test new therapeutic molecules in vivo.

### SUMMARY OF THE INVENTION

The present invention relates to a gallinaceous bird embryo, preferentially a chick or quail embryo, into which human neuroblastoma cells have been grafted at the level of the neural crests.

This graft is performed under appropriate conditions which allow these neuroblastoma cells to form tumors in the sympathetic ganglia and/or the adrenal gland medullae, approximately 48 h after the graft. The rapidity of preparation of this animal model is a great advantage of the invention.

This embryo is an animal study model for the human pediatric pathology that is neuroblastoma. This study model has the advantage of reproducing tumors in an environment similar to that of origin, namely an embryonic environment. In this model, the grafted human neuroblastoma cells migrate in the animal embryo in the same way as in the human pathology, and form tumors at the sites normally colonized by neuroblasts: sympathetic ganglia and adrenal gland medullae.

The present invention also relates to a process for preparing a gallinaceous bird embryo, comprising the following steps:
grafting human neuroblastoma cells at the level of the neural crests of a gallinaceous bird embryo, and
incubating the grafted embryo for at least 24 hours.

The present invention also relates to a process for monitoring a patient suffering from a neuroblastoma, comprising:

a) preparation of a first embryo according to the process above, with neuroblastoma cells from said patient at a time T1, and assessment of the tumorigenesis of the tumors developing in this first embryo,

b) preparation of a second embryo according to the process above, with neuroblastoma cells from said patient at a time T2, and assessment of the tumorigenesis of the tumors developing in this second embryo,

c) comparison between the tumorigenesis of the tumors developing in the first embryo and in the second embryo.

This model makes it possible to determine the characteristics of the tumors derived from the patients, in particular in terms of growth and dissemination capacity. These tumor cells, which may in particular be taken from a patient, and then grafted into a chick or quail embryo, may be used in particular for testing the efficacy of various therapies in vivo. This makes it possible to administer, to the patient, therapeutic molecules tested beforehand on cells from their own tumor, grafted into a gallinaceous bird embryo, thereby allowing a highly personalized therapeutic approach.

Thus, the invention also relates to a process for screening for therapeutic molecules intended for the treatment of a neuroblastic tumor in vivo, consisting of the following steps:

a) preparing embryos according to the process described above;

b) administering a candidate therapeutic molecule to these embryos;

c) assessing the tumorigenesis of the tumors present in the embryos after at least 24 hours of administration of said molecule.

### FIGURE LEGENDS

FIG. 1. Representation of the first stages of development of the chick embryo.

FIG. 2. Table of correspondence of the first stages of development of the quail (Japanese quail) and chick (chick) embryos.

FIG. 3. Diagrammatic representation of the process for preparing a gallinaceous bird embryo (Graft) and of the migration of the grafted cells (Analysis at 48 h).

FIG. 4. Location of the tumor foci 48 h after the graft of human neuroblastoma cells (dashed circles).

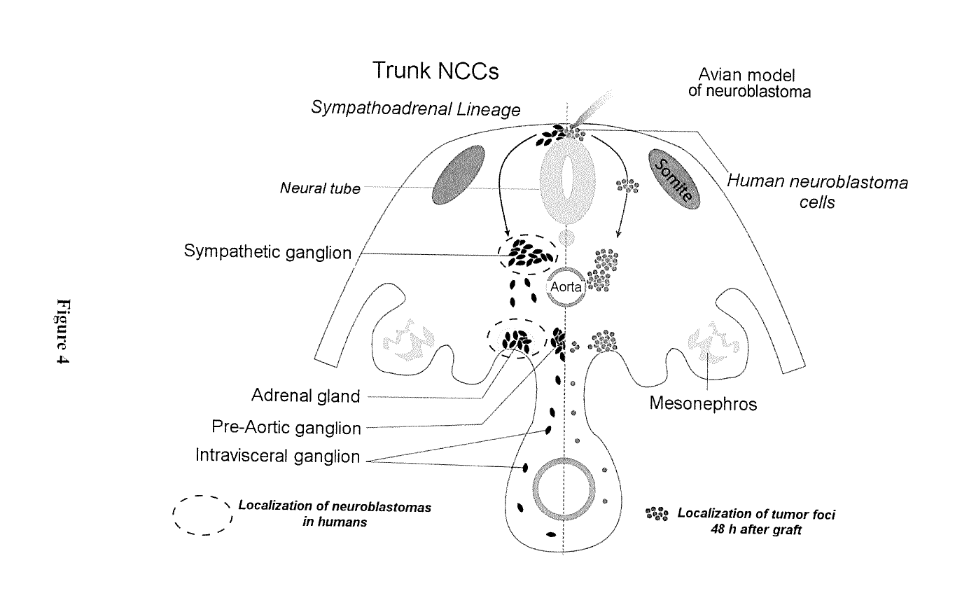

### DETAILED DESCRIPTION OF THE INVENTION

The gallinaceous bird embryo, in particular the chick or quail embryo, is an advantageous model for carrying out experiments in vivo, in particular for studying embryonic development and for xenotransplantation experiments. It is in fact inexpensive, very accessible and easy to handle. It is a model of choice for studying cell proliferation, differentiation and migration.

In particular, labeled cells, such as cells transformed with a gene encoding a fluorescent protein, have been used to visualize the migration of cells during development. Quail and chick chimeras developed by N. Le Douarin have been widely used to study cell migration during embryogenesis.

More recently, xenografts of human cells into the chick embryo have made it possible to study what happens to these human cells during the development of said embryo.

Patent application US 2013/0171680 describes the xenograft of malignant human hematopoietic cells in a chick embryo, these cells forming tumors within the embryo. This model makes it possible to test new therapeutic molecules, for determining their effect over time on these tumor cells introduced.

Carter et al. (Oncogenesis, 2012) have injected human neuroblastoma cells from tumors with a poor prognosis, overexpressing the MYCN oncogene, into the blood vessels of a chick embryo (stage 3 and 6 days). These neuroblastoma cells injected into the blood stream are directed to the sympathetic ganglia, but not to the adrenal gland. On contact with the embryonic microenvironment, the cells become reprogrammed into a more benign phenotype, in particular when they bind in neural tissues. This process does not therefore make it possible to reproduce the specifically localized tumors characteristic of the human disease, in the chick embryo.

The present invention relates to a gallinaceous bird embryo, preferentially a chick or quail embryo, into which human neuroblastoma cells have been grafted at the level of the neural crests, under conditions which allow these neuroblastoma cells to form tumors at the same sites as in the human disease, at the level of the sympathetic ganglia and the adrenal gland medullae.

The following terms are defined for better understanding of the invention:

The term “gallinaceous bird” denotes a bird of the order Galliformes (or gallinaceans) which comprises chicken, quail, turkeys, pheasants, peacocks, guinea-fowl, and other farmyard animals. Preferentially, the embryo will be from a chicken (Gallus gallus) or from a quail (Coturnix japonica), two species commonly used in the laboratory.

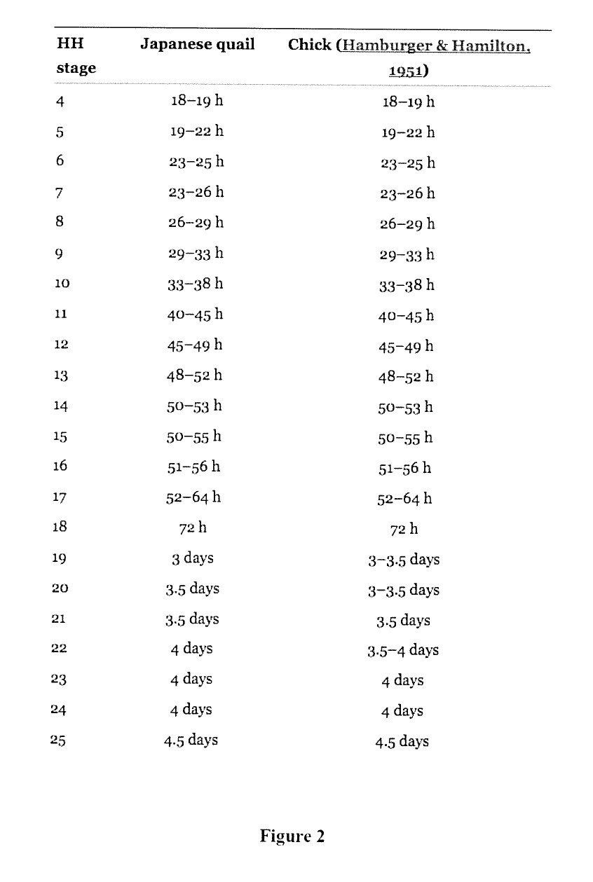

The term “gallinaceous bird embryo” denotes a fertilized gallinaceous bird egg in which an embryo develops normally, under appropriate conditions, in particular if it is placed in an incubator heated at a temperature of between 37° C. and 39° C. Several developmental stages have been listed and are indicated in FIG. 2, left-hand column for the quail embryo, right-hand column for the chick embryo.

The terms “embryo”, “gallinaceous bird embryo” and “grafted embryo” are used without distinction in the present application, and denote, for the purposes of the invention, a “chimeric embryo”, that is to say an embryo which has cells from at least two different individuals. Quail/chick chimeric embryos have been abundantly used to study the embryonic development of these species. In these experiments, whole fragments of an embryo were replaced with the equivalent fragments originating from a second embryo, this making it possible to determine what becomes of the grafted cells, that can be distinguished from the cells of the recipient embryo.

In the context of the present invention, the “recipient” or “receptor” embryo is a gallinaceous bird embryo which comprises all its own cells, and which also comprises human neuroblastoma cells, that have been grafted and have become an integral part of the embryo, following their acceptance as a graft within the recipient embryo.

It is understood that this chimeric embryo is not, in the real sense, a “chimer”, but denotes an animal study model in which the embryo is not intended to develop sufficiently to create an adult organism, but serves solely as a support for the human cells during a short period of time. In any event, it is understood that this gallinaceous bird embryo will not give rise to a living chimeric organism, but will be destroyed as soon as the study of what becomes of the grafted human cells has been completed.

The term “grafted” or “transplantation” denotes the introduction of exogenous cells into a receptor organism, at a precise site of the receptor organism, and in particular in specific tissues and not by injection into the blood stream. The present application relates in particular to “xenografts” since the cells introduced are from an organism that is from a species different from the recipient organism.

For the purposes of the invention, it is understood that these cells of external origin are not pluripotent cells, and that, in particular, these cells do not have a “stem cell” potential.

The term “neural crests” denotes, in the vertebrae embryo, a population of transient and multipotent cells generated from the most dorsal region of the neural tube. These cells migrate in the whole of the embryo during development and give rise to a large diversity of cell types in the adult. The neural crest is in particular responsible for all the glial cells and for most of the neurons of the peripheral nervous system.

Within embryonic neural crests, the neuroblasts are the progenitor cells which give rise to the sympathetic nervous system. The neuroblastoma is a malignant tumor derived from neuroblasts. As a result, it may be found over the entire length of the vertebral column and in the internal part of the adrenal gland. It is identified in particular in the sympathetic ganglia, which form part of the sympathetic nervous system, and in the adrenal medulla which is part of the adrenal glands. Advantageously, the adrenal glands exist both in mammals and in birds.

The term “human neuroblastoma cells” denotes tumor cells isolated from tumors resulting from the dysregulation of neuroblasts, in human children, or else immortalized cell lines from these tumor cells.

The invention relates to an embryo composed of a gallinaceous bird embryo, in particular a chick or quail embryo, onto which human neuroblastoma cells have been grafted, at the level of the neural crests, under suitable conditions which allow said cells to form tumors in the sympathetic ganglia and/or the adrenal gland medullae.

These “suitable conditions” allow the reproduction, in an animal model, of the human disease and in particular the formation of tumors at the specific sites (sympathetic ganglia and/or adrenal gland medullae) of tumor formation observed in patients suffering from neuroblastoma.

These “suitable conditions” are based essentially on the site of the graft, the developmental stage of the gallinaceous bird embryo, and the amount of human cells grafted.

The human neuroblastoma cells are grafted at the level of the neural crests of the gallinaceous bird embryo. According to one preferred aspect of the invention, the human neuroblastoma cells are grafted between somites 7 to 28 of the neural tube of the embryo, preferably between somites 12 to 24, preferably between somites 18 to 24 and more preferentially between somites 20 and 21 of the gallinaceous bird embryo. In particular, the incision enabling the graft of the cells is made in the roof of the neural tube, opposite somites 20 and 21.

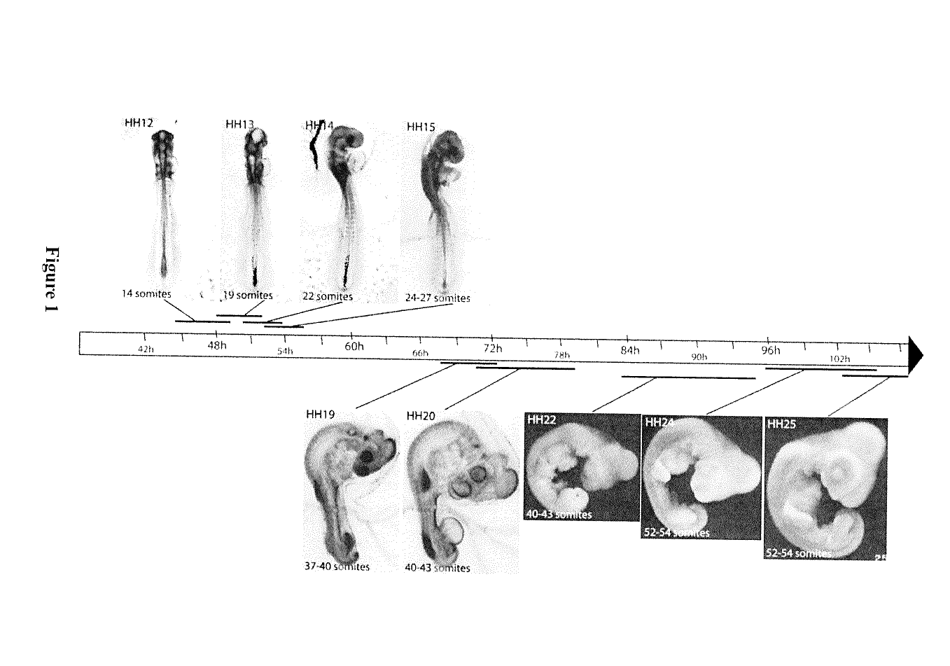

The neural tube comprises the primitive nervous system of the embryos. The somites denote the embryonic structures located on either side of the neural tube and the cord, and they are composed of repeating units along the anterior-posterior axis of the embryo. At the developmental stage between 48 and 55 hours post-fertilization, the gallinaceous bird embryo comprises 19 to 28 somites. A representation of the chick embryo at various developmental stages, comprising from 14 to 54 somites, is presented in FIG. 1.

The grafting of the human neuroblastoma cells into the gallinaceous bird embryo is carried out according to methods well known to those skilled in the art. The gallinaceous bird embryo is in fact easily accessible, after having made a small opening in the egg shell. In particular, the grafting of the human cells is carried out using a pressurized microinjector (Picopump PV830, World Precision Instruments). Other techniques for transplanting cells into the gallinaceous bird embryo have been described in the prior art, for example by Kulesa et al. (PNAS, 2006).

According to one preferred embodiment of the invention, the human neuroblastoma cells are grafted in an amount ranging from approximately 15 000 cells to approximately 75 000 cells per graft. In particular, the graft will comprise approximately 15 000 cells, approximately 20 000, approximately 25 000, approximately 30 000, approximately 35 000, approximately 40 000, approximately 45 000, approximately 50 000, approximately 55 000, approximately 60 000, approximately 65 000, approximately 70 000, or else approximately 75 000 human neuroblastoma cells.

The method which makes it possible to count the cells is well known to those skilled in the art. In particular, the number of cells grafted with the microinjector is determined before the grafting by counting, using a Malassez counting cell, the number of cells ejected from the capillary, for a given time and at a given pressure.

According to one preferential embodiment, at the time of the graft, the gallinaceous bird embryo, in particular the chick or quail embryo, is at a developmental stage between 48 and 55 hours post-fertilization, and preferably between 50 and 53 hours.

It is understood that the development of the embryo only begins when the embryo is incubated under correct conditions, in particular at a temperature of between 37° C. and 39° C. Thus, a development stage “between 48 and 55 hours” signifies that the egg has been maintained for this period under the optimal conditions for its development. A fertilized egg can be maintained at 14° C. before being placed under optimal conditions to its development; this waiting period at 14° C. is not to be taken into account in the duration indicated above.

At this development stage, the chick or quail embryo comprises between 19 and 28 somites. The development stage of the chick embryo is determined according to the criteria defined by Hamburger and Hamilton (1951, J. Morphol.), well known to those skilled in the art. FIG. 2 gives the equivalencies between the development stages of the chick and quail embryos.

Following the graft, the gallinaceous bird embryo is incubated for at least 24 hours according to the standard techniques, in a humidity-saturated incubator, at a temperature of between 37° C. and 39° C., preferably at 38.5° C.

As early as 24 hours of incubation, the first tumors are observed in the adrenal gland medullae and the sympathetic ganglia of the gallinaceous bird embryo.

According to one particular aspect of the invention, the embryo is incubated, after the human neuroblastoma cell graft, for at least 36 hours, at least 48 hours, at least 60 hours, at least 72 hours, at least 4 days, at least 5 days, or even up to hatching of the egg. According to one preferred aspect of the invention, the embryo is incubated for approximately 48 to 52 hours after the graft.

Thus, to distinguish the human neuroblastoma cells in the gallinaceous bird embryo, and in particular to monitor their dispersion and their multiplication capacity, the grafted cells are advantageously labeled.

Such a labeling may be carried out using dyes. The cells may in particular be labeled using vital dyes such as carbocyanides which have an affinity for cell membranes, into which they are incorporated, conferring a red fluorescence on the cells. Dyes of Carboxyfluorescein Succinimidyl Ester type (CFSE), which emit a green fluorescence when they react with intracellular proteins, may also be used.

According to one particular aspect of the invention, the grafted human neuroblastoma cells express a marker protein.

A marker protein denotes a protein encoded by an exogenous gene introduced into the cell by conventional genetic engineering methods, the expression of this gene being under the control of a promoter that is active in this cell, and this protein being visible, or being capable of reacting with a chemical reagent in order to become visible. Many marker proteins are known, such as Green Fluorescent Protein (GFP).

According to a first aspect of the invention, the human neuroblastoma cells are derived from at least one immortalized cell line.

Those skilled in the art are aware of several immortalized neuroblastoma cell lines, and in particular the following lines, used in the examples of the present application:
IGR-N-91 and LA-N-5: for reference, see the book “Neuroblastoma Cell Lines” by C J Thiele (1998).
SH-EP and SH-SY5Y: Cancer Res-1989-Ciccarone-219-25. These lines are sold by ATCC under the following names: SH-SY5Y (ATCC® ^{CRL}2266™) and SH-EP (ATCC® CRL-2269™).
IMR-32: Cancer Res-1970-Tumilowicz-2110-8. This line is sold by

ATCC under the following name: IMR-32 (ATCC® CCL-127™).

According to one particular aspect of the invention, several human neuroblastoma cell lines may be grafted into the embryo. According to one particular embodiment, in the gallinaceous bird embryo, the human neuroblastoma cells are derived from at least two immortalized cell lines, in particular two lines each expressing a different marker protein.

According to a second aspect of the invention, the human neuroblastoma cells grafted into the embryo are from a tumor from a patient. Said tumor, after removal by surgery, is dissected so as to isolate at least 15 000 neuroblastoma cells; these cells are then labeled with a vital dye, such as carbocyanides or Carboxyfluorescein Succinimidyl Ester (CFSE), and then grafted onto an embryo at the developmental stage between 48 and 55 hours post-fertilization.

The invention also relates to a process for preparing a gallinaceous bird embryo, in particular a chick or quail embryo, comprising grafting human neuroblastoma cells at the level of the neural crests of the embryo, under suitable conditions which allow said cells to form tumors in the sympathetic ganglia and/or the adrenal gland medullae of the gallinaceous bird embryo.

In particular, this process for preparing a gallinaceous bird embryo comprises the following steps:
grafting human neuroblastoma cells at the level of the neural crests of a gallinaceous bird embryo, and
incubating the grafted embryo for at least 24 hours.

According to one preferred aspect of the process, the human neuroblastoma cells are grafted between somites 7 to 28 of the neural tube of the embryo, preferably between somites 12 to 24 and more preferentially between somites 18 to 24.

Preferentially, the human neuroblastoma cells are grafted in a proportion of approximately 15 000 cells to approximately 75 000 cells/graft.

According to one preferred aspect of the process, the grafting is carried out on an embryo at a developmental stage of between 48 and 55 hours.

The incubation of the embryo is carried out under standard conditions, at a temperature of between 37° C. and 39° C. According to one particular aspect of the invention, the embryo is incubated, after the human neuroblastoma cells graft, for at least 36 hours, at least 48 hours, at least 60 hours, at least 72 hours, at least 4 days, at least 5 days, or even up to the hatching of the egg. According to one preferred aspect of the invention, the process comprises a step of incubating the grafted embryo for approximately 48 to 52 hours.

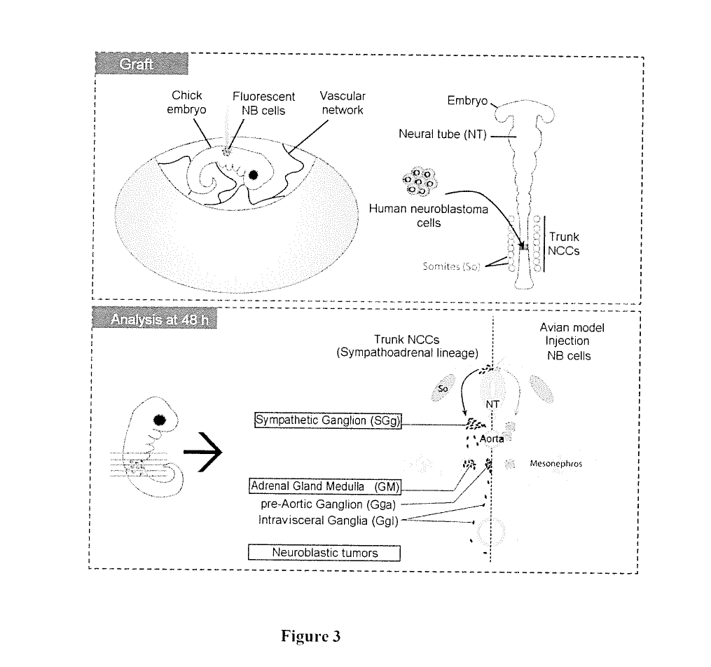

This process is represented diagrammatically in FIG. 3.

According to a first implementation of this process, the human neuroblastoma cells are derived from at least one immortalized cell line.

Thus, the invention relates to a process for preparing a gallinaceous bird embryo, comprising the following successive steps:
culturing at least one immortalized line of the neuroblastoma cells in a suitable medium;
grafting these cells, in a proportion of from 15 000 cells to 75 000 cells/graft, between somites 7 to 28, preferably between somites 12 to 24 and more preferentially between somites 18 to 24 of a gallinaceous bird embryo at a developmental stage of between 48 and 55 hours (post-fertilization),
incubating the grafted embryo for at least 24 hours.

The “suitable culture medium” denotes a culture medium suitable for the growth of the cells, for example DMEM medium or RPMI 1640 medium (Gibco), supplemented with antibiotics and comprising fetal calf serum.

Preferably, the cells of the immortalized line express a marker protein, or else are labeled with a dye, as previously presented.

The tumors in the sympathetic ganglia and/or the adrenal gland medullae appear as early as 24 hours, and are clearly identifiable after 48 hours of incubation of the grafted embryo. These tumors in the sympathetic ganglia and/or the adrenal gland medullae persist for at least 96 hours after the graft.

According to a second implementation of the process, the human neuroblastoma cells are from a tumor from a patient.

Thus, the invention relates to a process for preparing a gallinaceous bird embryo, comprising the following steps:
obtaining neuroblastoma cells from a patient, in particular by taking a sample of cells within their tumor;
grafting these cells between somites 7 to 28, preferably between somites 12 to 24 and more preferentially between somites 18 to 24 of a gallinaceous bird embryo at a developmental stage between 48 and 55 hours,
incubating the grafted embryo for at least 24 hours.

The human cells taken from the patient are counted and grafted in a proportion of at least 15 000 cells/graft, and preferably approximately 15 000 to approximately 75 000 cells per graft.

It will be possible to detect these cells in the recipient embryo, after they have been grafted. It will be possible for this detection to be carried out according to several processes well known to those skilled in the art, and in particular:
by visualization using an antibody which recognizes a membrane antigen specifically expressed by the neuroblastoma cells. By way of example, an antibody which recognizes disialoganglioside 2 (GD2), the surface antigen most commonly expressed by human neuroblastoma cells, may be used;
by genetic transformation of the neuroblastoma cells, prior to the grafting thereof, with a nucleic acid cassette encoding a fluorescent protein such as GFP (Green Fluorescent Protein), RFP (Red Fluorescent Protein) or mCherry (which fluoresces orange);
by staining the neuroblastoma cells, prior to the grafting thereof, with a vital cell dye. Vital dyes, such as carbocyanides which confer a red membrane fluorescence on the cells, or Carboxyflorescein Succinimidyl Ester (CFSE) which emits a green intercellular fluorescence, may be used.

The invention also relates to a process for monitoring a patient suffering from a neuroblastoma, comprising:

a) preparation of a first gallinaceous bird embryo according to the process described above, with neuroblastoma cells from said patient at a time T1, and assessment of the tumorigenesis of the tumors developing in this first embryo,

b) preparation of a second gallinaceous bird embryo according to the process described above, with neuroblastoma cells from said patient at a time T2, and assessment of the tumorigenesis of the tumors developing in this second embryo,

c) comparison between the tumorigenesis of the tumors developing in the first embryo and in the second embryo.

The tumors developing in the embryos are derived from the migration of the grafted human neuroblastoma cells. These tumors develop in the sympathetic ganglia and/or the adrenal gland medullae. These tumors consist of human neuroblastoma cells.

“The assessment of the tumorigenesis of the tumors” is carried out by several complementary approaches: after sampling of the tumors by microdissection, said tumors are subjected to various analyses:

biochemical and transcriptome studies; and

in vitro studies via the reculturing thereof.

These various analyses make it possible in particular to determine the following “tumorigenesis factors”:
localization of the tumor foci by histological analysis;
measurement of the tumor volume from 3-dimensional reconstructed images;

determination of the proliferation index in the tumor foci by detection of the Ki67 marker;
determination of the index of vascularization of the tumor foci by detection of angiogenesis markers;
determination of the index cell death in the tumor foci by detection of cell death events (DNA fragmentation, necrosis, cytochrome c release, pro-apoptotic protease activation);
analysis of the transcriptome and of the proteome of the tumors extracted in situ;
study of the cell behavior after reculturing.

The combined analysis of all these factors, well known to those skilled in the art, makes it possible to determine a “tumorigenesis index” which makes it possible to quantify the seriousness and the aggressiveness of the tumor. Indeed, all of these parameters make it possible to evaluate the state of differentiation of the neuroblastomic cells and also their capacity to proliferate and to disseminate in the organism. These parameters are an integral part of the anatomopathological classification of neuroblastomas which clinicians use as a basis for directing the therapeutic treatment.

Thus, it is possible to distinguish:
tumors which develop following the graft, in a gallinaceous bird embryo, of neuroblastoma cells, from a patient at a time T1, and
tumors which develop following the graft, in a gallinaceous bird embryo, of neuroblastoma cells from a patient at a time T2.

Such a process makes it possible to monitor, ex vivo, the development of a patient's disease, and in particular the tumorigenesis index of their tumor cells at a time T0 (for example, before the beginning of a treatment) and at a time T1, T2, T3 (for example, a few months after the beginning of the treatment of the patient).

The process may naturally be repeated the number of times required to monitor the progression of the disease in a given patient.

Finally, the invention relates to a process for screening for therapeutic molecules intended for the treatment of a neuroblastic tumor in vivo, consisting of the following successive steps:

a) preparing gallinaceous bird embryos according to one of the processes described above, from immortalized neuroblastoma cells or from neuroblastoma cells from a patient;

b) administering a candidate therapeutic molecule to these embryos;

c) assessing the tumorigenesis of the tumors present in the embryos after at least 24 hours of administration of said candidate molecule.

The term “candidate therapeutic molecule” is intended to mean a chemical or biological molecule which is potentially efficacious for treating the neuroblastoma.

The assessment of the tumorigenesis of the tumors is carried out by the approaches described above, after sampling of the tumors by microdissection. The comparison of the tumorigenesis of the tumors at time TO and of the tumors after at least 24 hours, in particular after 1 (T1), 2 (T2) or 3 (T3) days of administration of the molecule tested, makes it possible to determine the effect of the therapeutic molecule administered. Naturally, the administration of this molecule may be carried out for various times, in particular for at least 24 h, 48 h, 72 h, 96 h, and up to 5 days, 6 days, 7 days, 8 days, 9 days, 10 days, 11 days, 12 days, 13 days, 14 days, 15 days, 16 days, 17 days, 18 days, 19 days, 20 days, 21 days or up to the hatching of the egg, with the proviso that the tumors are always present in the gallinaceous bird embryo.

For a better understanding of the invention, a specific case is described in detail below:
a gallinaceous bird embryo is prepared with grafting of tumor cells from a patient;
after 48 h of incubation of the embryo, tumors are observed in this embryo;
a first sample of one of these tumors is taken (T0) and analyzed;
a therapeutic molecule is administered to this gallinaceous bird embryo by the usual techniques known to those skilled in the art, in particular by injection into the blood vessels of the embryo; the embryo is maintained under suitable temperature and humidity conditions for its development;
a second sample of the tumor is taken after 3 days of administration of the therapeutic molecule to the gallinaceous bird embryo (T3), and this sample is analyzed as described in detail above.

If the tumorigenesis index of the tumor sampled at T0 is high (serious and aggressive tumor), and if this tumorigenesis index of the tumor at T3 (after administration of the therapeutic molecule for 3 days) has decreased, going from “high” to “medium”, the experimenter will be able to conclude from this that the effect of the therapeutic molecule tested is positive.

### EXAMPLES

Materials and Methods

Cell Lines

The human neuroblastoma lines were obtained from ATCC (SH-EP, IMR-32, SH-SY5Y, LAN-5) or donated by Dr. J. Bénard (IGR-N-91, Institut Gustave Roussy, Villejuif, France). The SH-EP, IMR-32 and LAN-5 lines were cultured in RPMI 1640 medium (Gibco) and the IGR-N-91 and SH-SY5Y lines were cultured in DMEM medium (Gibco). Each medium was supplemented with 25 units/ml of penicillin-streptomycin (Gibco), 2.5 μg/ml of amphotericin B (Gibco) and 10% of fetal calf serum (Gibco).

Chick Embryos

The fertilized chicken (Gallus gallus) eggs were purchased from a local supplier (EARL Morizeau, Dangers, France) and maintained at 14° C. until use. The eggs were incubated at 38.5° C. for 52 hours in an incubator at a saturated level of humidity, so as to obtain embryos at the HH13 to HH16 developmental stage.

Human Tumor Line Grafts in the Chick Embryo

5×10⁶ tumor cells were harvested and were suspended in 30 μl of medium. After 52 hours of incubation at 38.5° C., a window was cut in the shell in order to visualize and access the embryo. The vitelline membrane was cut at the level of the neural tube and a wound was made in the roof of the neural tube, opposite somites 20 and 21. The cells were inserted into a glass microcapillary and deposited using a pressurized microinjector (Picopump PV830, World Precision Instruments). The cells were thus grafted at the level of the dorsal roof of the neural tube on a zone corresponding to 1 to 2 somites. The eggs were then put back in the incubator at 38.5° C. for 52 hours.

Chick Embryo Sections

The embryos were harvested and fixed in 4% paraformaldehyde overnight at 4° C. Depending on the type of analysis desired, the embryos were cut to produce transverse and sagittal sections. The sections were maintained in PBS at 4° C. in the dark until use. The localization of the tumors was studied by various immunolabelings and detection of the fluorescence of the tumor cells transduced beforehand so as to express GFP or RFP (marker proteins).

Tumor Sampling In Situ

Analysis The tumors are sampled by microdissection and subjected to various analyses:
biochemical and transcriptome studies (characterization and search for known or new molecular markers), and
in vitro studies via the reculturing thereof.

Taking Images and Processing

The sections were analyzed using a confocal microscope (Olympus IX81). The entire image of the section was reconstituted using the XuvTools software.

The tumorigenesis was assessed by means of various analyses:
determination of the localization of the tumor foci by histological analysis;
measurement of the tumor volume from the 3-dimensional reconstructed images;
determination of the proliferative index in the tumor foci by detection of the Ki67 marker;
determination of the index vascularization of the tumor foci by detection of angiogenesis markers;
determination of the index cell death in the tumor foci by detection of cell death events (DNA fragmentation, necrosis, cytochrome c release, pro-apoptotic protease activation);
analysis of the transcriptome and of the proteome of the tumors extracted in situ;
study of the cell behavior after reculturing.

RESULTS

Five human stage-4 neuroblastoma lines: IGR-N-91, Lan5, SH-SY5Y, SH-EP, and IMR32, were grafted at the level of the dorsal roof of the spinal cord of chick embryos, which is the site of the sympathoadrenal neural crests. In each embryo, a single cell line was introduced, in a proportion of approximately 15 000 to 75 000 cells/graft. 48 hours after the graft, in 60% to 95% of the embryos depending on the line grafted, the fluorescent cells left the graft site and took a ventral path, similar to that taken by endogenous neural crest cells. The formation of tumor masses was observed exclusively in the sympathetic ganglia, in the adrenal gland medullae and/or between the spinal cord and the dorsal spinal ganglia. The localization of these tumor foci is identical to the neuroblastoma development sites in children.

72 h and 96 h after the graft, these tumor masses are maintained, amplified and are associated with secondary foci mainly detected at the level of the retroperitoine, a major site of development of neuroblastic metastases in humans. Interestingly, this behavior appears to be specific for the neuroblastoma lines. Indeed, fluorescent melanoma or pulmonary carcinoma cells grafted into the chick embryo according to an identical protocol do not migrate according to a “ventral” path and do not become established in the sympathoadrenal derivatives.

Furthermore, the possibility of grafting, in the same chick embryo, two neuroblastoma lines expressing a different fluorescent protein was studied. Thus, the tissue localization of the cells of each of the lines can be observed distinctly, 48 h after the graft. This makes it possible to directly compare, while avoiding any bias related to a possible inter-embryo variability, the behavior of two neuroblastoma lines. For example, cells which have been subjected to a prior treatment of the graft may be directly compared with their non-treated homologs. Likewise, any behavioral differences in tumor samples from patients before and after drug treatments may be easily measured.

### LITERATURE REFERENCES

### Patents

US 2013/0171680

### NON-PATENTS

Weiss W A, Aldape K, Mohapatra G, Feuerstein B G, Bishop J M. Targeted expression of MYCN causes neuroblastoma in transgenic mice. EMBO J. 1997 Jun. 2; 16(11):2985-95.
Zhu S, Lee J S, Guo F, Shin J, Perez-Atayde A R, Kutok J L, Rodig S J, Neuberg D S, Heiman D, Feng H, Stewart R A, Wang W, George R E, Kanki J P, Look A T. Activated ALK collaborates with MYCN in neuroblastoma pathogenesis. Cancer Cell. 2012 Mar. 20; 21(3):362-73.
R Carter, D Mullassery, V See, S Theocharatos, B Pizer, PD Losty, E Jesudason and D J Moss. Exploitation of chick embryo environments to reprogram MYCN-amplified neuroblastoma cells to a benign phenotype, lacking detectable MYCN expression. Oncogenesis. 2012 Aug. 27; 1:e24
Kulesa P M, Kasemeier-Kulesa J C, Teddy J M, Margaryan N V, Seftor E A, Seftor R E, Hendrix M J. Reprogramming metastatic melanoma cells to assume a neural crest cell-like phenotype in an embryonic microenvironment. Proc Nati Acad Sci U S A. 2006 Mar. 7; 103(10):3752-7.
Hamburger V, Hamilton H L. A series of normal stages in the development of the chick embryo. J Morphol. 1951 January; 88(1):49-92.
Thiele C J. Neuroblastoma: In (Ed.) Masters, J. Human Cell Culture. Lancaster, UK: Kluwer Academic Publishers. 1998, Vol 1, p 21-53.
Ciccarone V, Spengler B A, Meyers M B, Biedler J L, Ross R A. Phenotypic diversification in human neuroblastoma cells: expression of distinct neural crest lineages. Cancer Res. 1989 Jan. 1; 49(1):219-25.
Tumilowicz J J, Nichols W W, Cholon J J, Greene A E. Definition of a continuous human cell line derived from neuroblastoma. Cancer Res. 1970 August; 30(8):2110-8.

1. Gallinaceous bird embryo into which human neuroblastoma cells have been grafted at the level of the neural crests.

2. Gallinaceous bird embryo according to claim 1, characterized in that the human neuroblastoma cells are grafted between somites 7 to 28 of the neural tube of the embryo.

3. Gallinaceous bird embryo according to claim 1, characterized in that the human neuroblastoma cells are grafted in an amount ranging from approximately 15 000 cells to approximately 75 000 cells per graft.

4. Gallinaceous bird embryo according to claim 1, characterized in that the grafting is carried out on an embryo at a development stage of between 48 and 55 hours.

5. Gallinaceous bird embryo according to claim 1, characterized in that the embryo is incubated for at least 24 hours after the graft.

6. Gallinaceous bird embryo according to claim 1, characterized in that the human neuroblastoma cells express a marker protein.

7. Gallinaceous bird embryo according to claim 1, characterized in that the human neuroblastoma cells are derived from at least one immortalized cell line.

8. Gallinaceous bird embryo according to claim 7, characterized in that the human neuroblastoma cells are derived from two immortalized cell lines each expressing a different marker protein.

9. Gallinaceous bird embryo according to claim 1, characterized in that the human neuroblastoma cells are from a tumor from a patient.

10. Process for preparing a gallinaceous bird embryo, comprising the following steps:
grafting human neuroblastoma cells at the level of the neural crests of a gallinaceous bird embryo, and
incubating the grafted embryo for at least 24 hours.

11. Process according to claim 10, characterized in that the human neuroblastoma cells are grafted between somites 7 to 28 of the neural tube of the embryo, preferably between somites 12 to 24 and more preferentially between somites 18 to 24.

12. Process according to claim 10, characterized in that the human neuroblastoma cells are grafted in an amount ranging from approximately 15 000 cells to approximately 75 000 cells per graft.

13. Process according to claim 10, characterized in that the grafting is carried out on an embryo at a development stage of between 48 and 55 hours.

14. Process according to claim 10, characterized in that the human neuroblastoma cells are derived from at least one immortalized cell line.

15. Process according to claim 10, characterized in that the human neuroblastoma cells are from a tumor from a patient.

16. Process for monitoring a patient suffering from a neuroblastoma, comprising:
a) preparation of a first embryo according to the process of claim 15, with neuroblastoma cells from said patient at a time T1, and assessment of the tumorigenesis of the tumors developing in this first embryo,
b) preparation of a second embryo according to the process of claim 15, with neuroblastoma cells from said patient at a time T2, and assessment of the tumorigenesis of the tumors developing in this second embryo,
c) comparison between the tumorigenesis of the tumors developing in the first embryo and in the second embryo.

17. Process for screening for therapeutic molecules intended for the treatment of a neuroblastic tumor, consisting of the following steps:
a) preparing embryos according to the process of claim 10;
b) administering a candidate therapeutic molecule to these embryos;
c) assessing the tumorigenesis of the tumors present in the embryos after at least 24 hours of administration of said candidate molecule
